(19) United States
(12) Patent Application Publication
(10) Pub. No.: US 2025/0389926 A1
(43) Pub. Date: Dec. 25, 2025

(54) LENS STRUCTURES AND OPTICAL SYSTEMS

(71) Applicant: ALPHAMICRON INCORPORATED, Kent, OH (US)

(72) Inventors: Eui-Yeul Park, Hudson, OH (US); Christine Baker, Munroe Falls, OH (US); Bahman Taheri, Shaker Heights, OH (US)

(73) Assignee: ALPHAMICRON INCORPORATED, Kent, OH (US)

(21) Appl. No.: 19/249,658

(22) Filed: Jun. 25, 2025

Related U.S. Application Data
(60) Provisional application No. 63/664,009, filed on Jun. 25, 2024.
(60) Provisional application No. 63/672,093, filed on Jul. 16, 2024.

(51) Int. Cl. G02B 7/02; G02B 1/18; G02C 7/08

(52) U.S. Cl. CPC G02B 7/021; G02B 1/18; G02C 7/083; G02C 7/088

(57) ABSTRACT

A lens structure (101) includes a light transmissive front lens (110) spaced away from a rear optical element (130) by a pressure-relieving attachment structure (120) provided around a viewing area perimeter of a front surface (139) of the rear optical element. The pressure-relieving attachment structure further contacts a rear surface (111) of the front lens to form a gap (140) between the front lens and the rear optical element.

### CROSS-REFERENCE TO RELATED APPLICATIONS

This application claims priority to, and any other benefit of, U.S. Provisional Patent Application Ser. No. 63/672,093, entitled LENS STRUCTURES AND OPTICAL SYSTEMS, filed Jul. 16, 2024, and to U.S. Provisional Patent Application Ser. No. 63/664,009, entitled FLEXIBLE ELECTRONIC EYEWEAR SYSTEM, filed Jun. 25, 2024, the entire disclosures of which is fully incorporated herein by reference.

### TECHNICAL FIELD

The present disclosure relates to lens structures and optical systems that may be used in functionalized eyewear, that in some cases, may also be robust to impact. Such eyewear may, for example, be used by military personnel, law enforcement, sports enthusiasts, or others at risk of having eyewear receiving undesired impact from accidents, projectiles, or the like.

### BACKGROUND

Some safety eyewear may include a lens portion that is designed primarily to prevent or reduce damage to a user's eyes, e.g., from wind, dust, airborne debris, projectiles, UV rays, bright light, or the like. Such safety eyewear may fit over a user's prescription eyewear, but these designs are bulky in nature. The eyewear can be functionalized to include, e.g., a prescription lens or even an electronically active lens, which is typically located behind the lens intended for eye safety. When the functionalized eyewear receives a relatively high impact, however, the functionalized rear lens is easily damaged or dislodged so that it no longer functions.

Thus, there is a desire for functionalized eyewear that maintains functionality even after receiving an impact force, or that includes components that can be easily replaced.

### SUMMARY

According to some aspects, the present disclosure provides a lens structure that includes a light transmissive front lens spaced away from a rear optical element by a pressure-relieving attachment structure provided around a viewing area perimeter of a front surface of the rear optical element. The pressure-relieving attachment structure further contacts a rear surface of the front lens to form a gap between the front lens and the rear optical element.

In some aspects, an eyewear system includes the lens structure, and when the front lens is impact resistant, the eyewear system meets the requirements of the MIL-PRF-31013 standard, the MIL-DTL-43511D standard, or the ANSI Z87.1 standard.

According to some aspects, an optical system is provided. The optical system may include a frame having a frame opening, a light transmissive front lens extending across the frame opening and attached to the frame by a front attachment element, a rear optical element extending across the frame opening and attached to the frame by a rear attachment element. The rear optical element is spaced away from the front lens by a gap. The optical system further includes a pressure-relieving structure responsive to pressure changes within the gap. The pressure-relieving structure may include i) a pressure-relieving attachment structure corresponding to the front attachment element ii) a pressure-relieving attachment structure corresponding to the rear attachment element iii) a pressure-relieving frame element, or iv) any combination of (i)-(iii).

### BRIEF DESCRIPTION OF DRAWINGS

FIG. 1A is a front view schematic of non-limiting example of a lens structure that may be used in a functionalized eyewear system.

FIG. 1B is a cross-sectional schematic of the lens structure from FIG. 1A taken along cutline B-B.

FIG. 1C front view schematic of another non-limiting example of a lens structure that may be used in a functionalized eyewear system.

FIG. 1D is a front view schematic of another non-limiting example of lens structures that may be used in a functionalized eyewear system.

FIG. 2 is a cross-sectional schematic of a lens structure receiving an impact.

FIGS. 3A and 3B are cross-sectional schematics illustrating a non-limiting example of a pressure-relieving attachment structure based on a HAL fastener.

FIG. 4 is a cross-sectional schematic of another non-limiting example of a pressure-relieving attachment structure.

FIG. 5 is a cross-sectional schematic of another non-limiting example of a pressure-relieving attachment structure.

FIGS. 6A and 6B are front and top views of a prior art eyewear system using a UPLC (Universal Prescription Lens Carrier).

FIG. 7A is a photograph of a non-limiting example of a lens structure after ballistics testing.

FIG. 7B is a photograph of a comparative lens structure (not according to the present disclosure) after ballistic testing.

FIGS. 8A-8G are various views of some non-limiting examples of optical systems according to the present disclosure.

### DETAILED DESCRIPTION

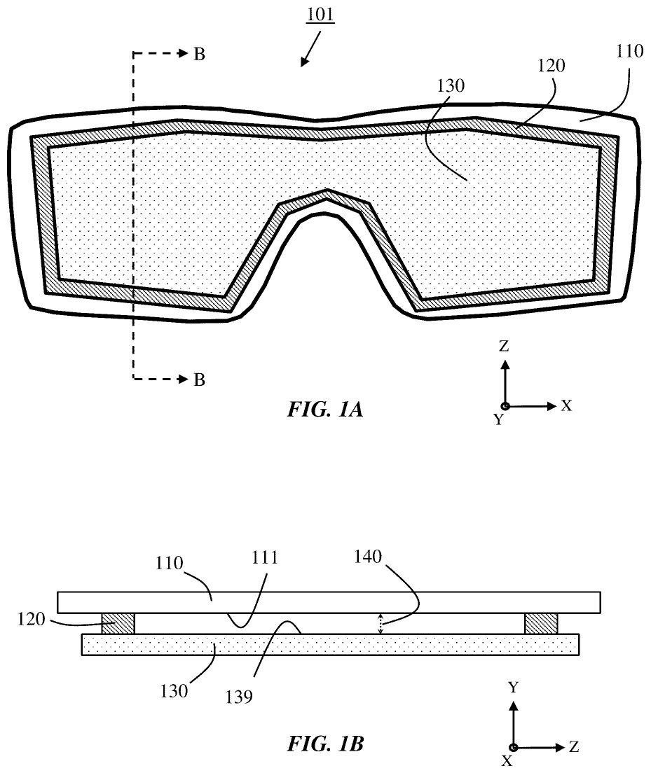

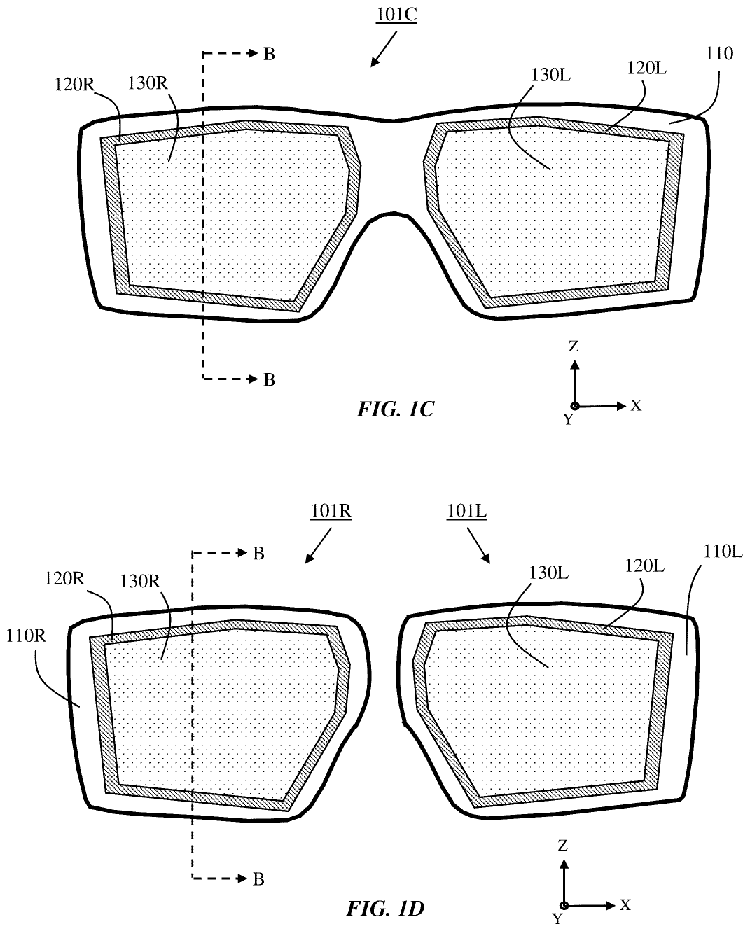

FIG. 1A is a front view schematic of non-limiting example of a lens structure 101 that may be used in a functionalized eyewear system. FIG. 1B is a cross-sectional schematic of the lens structure 101 taken along cutline B-B of FIG. 1A. For additional perspective, XYZ coordinate axes are shown. Lens structure 101 includes a light transmissive front lens 110 spaced away from rear optical element 130 by a pressure-relieving attachment structure 120. The pressure-relieving attachment structure is provided around a viewing area perimeter of a front surface 139 of the rear optical element. The pressure-relieving attachment structure further contacts a rear surface 111 of the front lens 110 to form a space or gap 140 between the front lens and rear optical element. Note that a “light transmissive” lens is one that transmits at least 5% of incident light within at least a portion of the visible light spectrum of 400 nm-700 nm, alternatively, at least 10%, at least 20%, at least 30%, at least 40%, at least 50%, at least 60%, at least 70%, at least 80%, at least 90%, or at least 95%. In some cases, a light transmissive lens may transmit at least 5% of light across the entire visible light spectrum, but in other cases, it may transmit at least 5% of light for only a range within (a portion) the visible light spectrum (e.g., within 400 nm-450 nm, 450 nm-500 nm, 500 nm-550 nm, 550 nm-600 nm, 600 nm-650 nm, 650 nm-700 nm, or any combination of ranges thereof).

Although not illustrated, the lens structure 101 may be attached to an eyewear frame, form part of a visor for a helmet, be incorporated into a face mask, or any other suitable functionalized eyewear system. In some cases, the lens structure may be attached to the frame by the front lens, by the rear optical element, or both.

FIG. 1C is front view schematic of another non-limiting example of a lens structure that may be used in a functionalized eyewear system. Lens structure 101C is similar to lens structure 101, but rather than a single rear optical element extending over both eyes, right rear optical element 130R is attached to a right portion of the front lens 110 by right pressure-relieving attachment structure 120R. Similarly, left rear optical element 130L is attached to a left portion of the front lens 110 by left pressure-relieving attachment structure 120L. In this case, the viewing area perimeter may correspond to either the left or right eye perimeters where the respective pressure-relieving attachment structures are provided. Cutline B-B may correspond to a similar cross-sectional structure as shown in FIG. 1B.

FIG. 1D is a front view schematic of another non-limiting example of a lens structure that may be used in a functionalized eyewear system. Here, there are separate right and left lens structures, 101R and 101L, respectively, which may be attached to an eyewear frame, form part of a visor for a helmet, be incorporated into a face mask, or provided in any other suitable functionalized eyewear system. Right lens structure 101R includes a light transmissive front lens 110R spaced away from rear optical element 130R by a pressure-relieving attachment structure 120R. Similarly, left lens structure 101L includes a light transmissive front lens 110L spaced away from rear optical element 130L by a pressure-relieving attachment structure 120L. Cutline B-B may correspond to a similar cross-sectional structure as shown in FIG. 1B.

While the following discussion is generally directed to lens structure 101, it will be appreciated that the concepts may also be applied to lens structures 101C, 101L, and 101R, and other lens structures.

The front lens may be made from glass, plastic, or a composite. In some examples, the front lens may be impact resistant. In some cases, the front lens 110 may be made from polycarbonate or some other impact-resistant, light-transmissive material. In some cases, lens structure 101 or a functionalized eyewear system incorporating the lens structure 101 may meet the requirements of at least one of the MIL-PRF-31013 standard, the MIL-DTL-43511D standard, or the ANSI Z87.1 standard. Front lens 110, may be clear or tinted or include a polarizer. In some cases, the front lens may be configured to absorb or reflect narrowband radiation, such as laser light or certain LED lights. In some cases, the rear surface and/or the front surface of the front lens may include an antifog coating,

The rear optical element 130 may be passive (non-electrically active) or active (electronically active). In some examples, the rear optical element may include a prescription lens, a polarizer, or a photochromic dye. In some cases, the rear optical element may be configured to absorb or reflect narrowband radiation, such as laser light or certain LED lights. Note that “narrowband radiation” herein refers to radiation having a wavelength bandwidth less than 88 nm, alternatively less than 80, 70, 60, 50, 40, 30, 20, 15, 10, 9, 8, 7, 6, 5, 4, 3, 2, or 1 nm. In some cases, bandwidth may correspond to a full-width-at-half-max (FWHM) of a spectrum of relative radiant power vs. wavelength. In some cases, rear optical element may include an electronically active lens.

The term “electronically active lens” refers to an electronically active optical element, such a liquid crystal cell, an electro-chromic cell or any other electronically active optical device whose optical properties can be altered by application of a current or voltage. Some non-limiting examples include an electronically active tinting lens (e.g., E-Tint®), a virtual reality (VR) device, an augmented reality (AR) device, a near-eye display, or the like. The electronically active lens may in some cases include an electronic optical device laminated or otherwise attached to a passive (non-electronic) lens carrier, e.g., made from glass or plastic. If used, a lens carrier may optionally be a prescription lens. The lens carrier may be clear or tinted or act as a polarizer.

In some cases, the front lens or rear optical element may be flexible. In some cases, a flexible front lens or a flexible rear optical element may be one that can be flexed under a bending force such that an edge relative to a center point of the lens or optical element can be flexed by a flexing angle of at least 5° relative to the edge position in the absence of the bending force, alternatively at least 10°, 15°, 20°, 25°, 30°, 45°, 60°, 75°, 90°, 120°, or 150°, or even up to 180°. In some cases, such flexing may be reversible (the lens or optical element can be reversibly flexed). By “reversibly flexed” or “reversible flexibility” it is meant that it can be flexed between a first position and a second position at least twice (alternatively at least 10 times) without functional damage to the lens or optical element, i.e., it still operates as intended after such flexing.

In some cases, the rear optical element 130 may have more flexibility than the front lens 110, e.g., under a similar force, the rear optical element 130 may bend more than the front lens 110. Such comparison may be made using a three-point bend test apparatus. In some examples, the front surface and/or the rear surface of the rear optical element may include an antifog coating.

In some cases, an electronically active lens may include one or more of a liquid crystal (LC) device, a variable transmission optical device, an electrochromic optical device, a reversible metal electrodeposition device, a switchable polarizer device, a graduated electrooptic device, an electronic lensing device (i.e., that alters the direction or focus of incident light), or a switchable light reflective device. Any one or more LC configurations are contemplated, including Twisted Nematic (TN), Hyper Twisted Nematic (HTN), Super Twisted Nematic (STN), Film Compensated Super Twisted Nematic (FSTN), Wide View Twisted Nematic (WVTN), Vertically Aligned Nematic liquid crystal (VA or VAN), In-Plane Switching (IPS) and Fringe Field Switching (FFS), Multidomain Vertically Aligned (MVA and PVA), Axially Symmetric Vertically Aligned aka Advanced Super View (ASV), Amplified Intrinsic Fringe-Field Multidomain Vertically Aligned (AIFF MVA), and any other LC configuration known in the art. Any of the foregoing devices can also be referred to herein as a type of electronically active optical element.

In some cases, the electronically active lens may include a liquid crystal device having a liquid crystal host and a dichroic or photodichroic dye guest, such as those described in U.S. Pat. No. 6,239,778 or US2022039226, the entire contents of which are incorporated herein by reference for all purposes.

As some non-limiting examples, an electronically active optical element substrate may include (alone or in combination with other materials) a polycarbonate (PC), a polycarbonate and copolymer blend, a polyethersulfone (PES), a polyethylene terephthalate (PET), cellulose triacetate (TAC), a polyamide, p-nitrophenyl butyrate (PNB), a polyetheretherketone (PEEK), a polyethylene naphthalate (PEN), a polyetherimide (PEI), polyarylate (PAR), a polyvinyl acetate, a cyclic olefin polymer (COP) a polyester, a polyurethanes, a polysilicone, a polyacrylate, a polypropylene, a polyethylene, a polystyrene, a polyvinyl chlorides, a polylactic acid, an ABS polymer, or some other polymeric material having the desired properties. An electronically active optical element substrate may include a composite of materials, for example, a polymeric material in combination with a glass, ceramic, or other inorganic additives. In some non-limiting examples, the electronically active optical element substrate may be glass. In some cases, flexible glass including materials such as Corning® Willow® Glass and the like can be used. An electronically active lens substrate may include multiple materials or have a multi-layer structure.

While not limiting, in some cases, gap 140 may have an average thickness in a range of 0.05-0.1 mm, 0.1-0.2 mm, 0.2-0.3 mm, 0.3-0.4 mm, 0.4-0.5 mm, 0.5-0.6 mm, 0.6-0.7 mm, 0.7-0.8 mm, 0.8-0.9 mm, 0.9-1.0 mm, 1.0-1.2 mm, 1.2-1.4 mm, 1.4-1.6 mm, 1.6-1.8 mm, 1.8-2.0 mm, 2.0-2.2 mm, 2.2-2.4 mm, 2.4-2.6 mm, 2.6-2.8 mm, 2.8-3.0 mm, 3.0-3.5 mm, 3.5-4.0, 4.0-5.0 mm, 5.0-7.0 mm, 7.0-10 mm, 10-15 mm, 15-20 mm, or any combination of ranges thereof.

While not limiting, in some examples, an area formed by the perimeter of pressure-relieving attachment structure 120 may be in a range of 1-2 cm², 2-5 cm², 5-10 cm², 10-20 cm², 20-30 cm², 30-40 cm², 40-50 cm², 50-70 cm², 70-100 cm², 100-150 cm², 150-200 cm², 200-300 cm², 300-400 cm², 400-500 cm², 500-700 cm², 700-1000 cm², or any combination of ranges thereof.

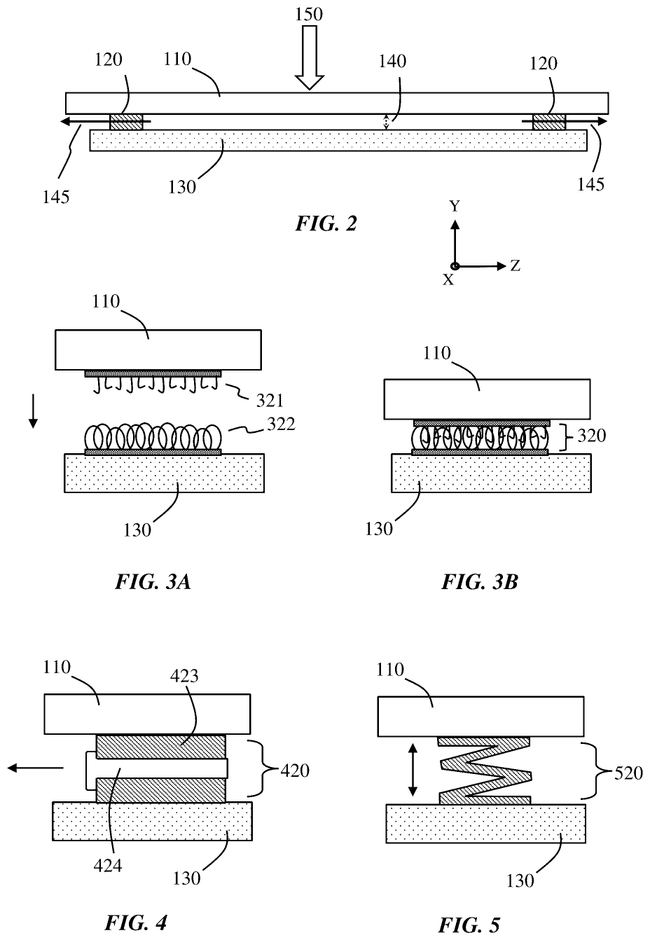

The pressure-relieving attachment structure 120 acts to attach the rear optical element to the front lens. The pressure-relieving attachment structure is designed so that it can facilely relieve sudden changes in gas pressure within gap 140 that may occur when the lens structure receives an impact. FIG. 2 is a cross-sectional schematic of lens structure 101 that may be similar to that shown in FIG. 1B, but now receiving a force or impact 150, e.g., from a projectile, an accident, an explosion shockwave, or the like. The impact 150 can create a sudden internal pressure within gap 140 through compressive forces. In some cases, the internal pressure may be in the form of a shock wave. Conventional functionalized eyewear typically employs a permanent adhesive seal in the perimeter area that does not have pressure relieving properties. The sudden pressure change or shock wave is received by the rear optical element (typically not impact resistant) which can cause irreversible damage and/or its detachment from the front lens. Such detachment can potentially cause damage to the user's eyes. Surprisingly, stronger adhesives do not help solve these deficiencies. It has been unexpectedly found that a pressure-relieving attachment structure can provide a more durable system

Referring again to FIG. 2, upon impact 150 (for example, an impact as defined in the MIL-PRF-31013 standard, the MIL-DTL-43511D standard, or the ANSI Z87.1 standard.), gas 145 (e.g., air) from the gap may be vented through the pressure-relieving attachment structure 120 at a venting rate sufficient to maintain attachment and preserve functionality of rear optical element 130. In some cases, the pressure-relieving attachment structure may be described as including a gas-venting structure. For example, if a pressure within gap 140 is increased from pressure P1 to pressure P2, the pressure-relieving structure may be capable of reducing pressure (e.g., by venting gas) from P2 to less than (P2−P1)/2 within a time T, wherein T is less than 1 sec, alternatively, less than 0.5 sec, 0.2 sec, or 0.1 sec. Time T may sometimes be referred to as a pressure-relief half-life. In some cases, a ratio of P2 to P1 may be at least 1.05, alternatively at least 1.1, at least 1.2, at least 1.5, at least 2, or at least 5.

In some examples, the pressure-relieving attachment structure includes a hook-and-loop set of materials (“HAL fastener”), commonly referred to as Velcro® or the like. FIGS. 3A and 3B are cross-sectional schematics illustrating a pressure-relieving attachment structure 320 based on a HAL fastener according to some non-limiting examples. On the rear side of the front lens 110, a “hook” structure 321 is provided, e.g., by an adhesive tape on a side opposite that of the hooks. The corresponding “loop” structure 322 is provided on the front surface of rear optical element 130, e.g., by an adhesive tape on a side opposite that of the loops. The front lens and rear optical element are brought together to form the pressure-relieving attachment structure 320, which in this case may also be referred to as HAL fastener 320. Note that the positions of hook structure 321 and loop structure 322 may be reversed from what is shown.

Unlike an adhesive seal, the HAL fastener 320 allows gas-venting upon impact. The rate of venting or gas permeation can be controlled in part by the density of hook-and-loop structures and the overall height. The HAL fastener may also allow some pressure relief in the Y direction (a type of pressure-flexing structure as discussed below). That is, the HAL fastener may be compressed or stretched to partially absorb an impact force thereby reducing stresses transferred to the rear optical element. As an additional benefit, HAL fasteners are typically reversibly attachable/detachable, which can allow easy replacement of a rear optical element 130 or front lens 110. In some cases, the front lens may be a replaceable protective lens that protects the rear optical element, and which is easily removed and replaced, e.g., if it gets scratched or damaged. For example, a rear optical element may be attached (optionally permanently) to a frame and the front lens is a protective replaceable protective lens. In some cases, the rear optical element/frame may be rather expensive, but the replaceable protective lens may be made from inexpensive materials.

FIG. 4 is a cross-sectional schematic of another pressure-relieving attachment structure according to some non-limiting examples. Pressure-relieving attachment structure 420 may include an attachment matrix 423 (e.g., an adhesive) in which are provided one or more pressure relief elements 424, e.g., one or more pressure relief valves. The pressure relief element(s) may be provided at various locations along the perimeter. Upon impact, if a pressure exceeds a predetermined limit, the pressure relief element may open, break, or be ejected to allow gas within the gap to vent.

FIG. 5 is a cross-sectional schematic of another pressure-relieving attachment structure according to some non-limiting examples. Pressure-relieving attachment structure 520 may include a bellows or other flexing structure to allow facile flexing in the Y axis. Such a pressure-relieving attachment structure may be referred to herein as including a pressure-flexing structure. This pressure-flexing structure may be compressed or stretched to partially absorb an impact force thereby reducing stresses transferred to the rear optical element.

### Uses

In some non-limiting examples, the present lens structures may be incorporated into eyewear systems that may be worn by an aviator, an armed forces member, a law enforcement officer, a skier, a sports enthusiast, a motorcyclist, or an ATV operator. In some cases, the present lens structures described herein may instead be incorporated into windows, windshields, or sunroofs.

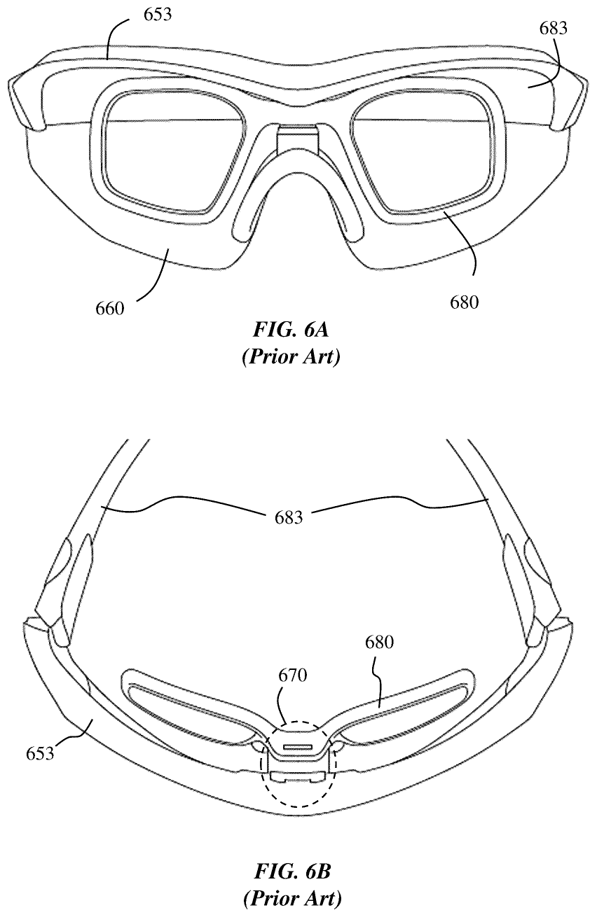

Functionalized eyewear systems using the present lens structures can offer a much lower profile than some conventional functionalized eyewear systems. The eyewear system's profile may correspond to how far the system extends from the wearer's face, e.g., a distance from the user's eye(s) to the lens furthest away. For example, FIGS. 6A and 6B are front and top views of a prior art eyewear system having a so-called UPLC (Universal Prescription Lens Carrier) having a front lens 660 attached to a front frame 653 and temples 683 attached to the front frame. The UPLC includes a bridge extension 670 to which is attached the rear prescription lens assembly 680. In the UPLC design, the front lens sits far out in front of the rear optical elements. This high profile can have disadvantages, e.g., when the eyewear assembly is designed to fit under a visor or a helmet. The spacing of the front lens and rear optical element(s) of the present disclosure can be made much closer.

### Example

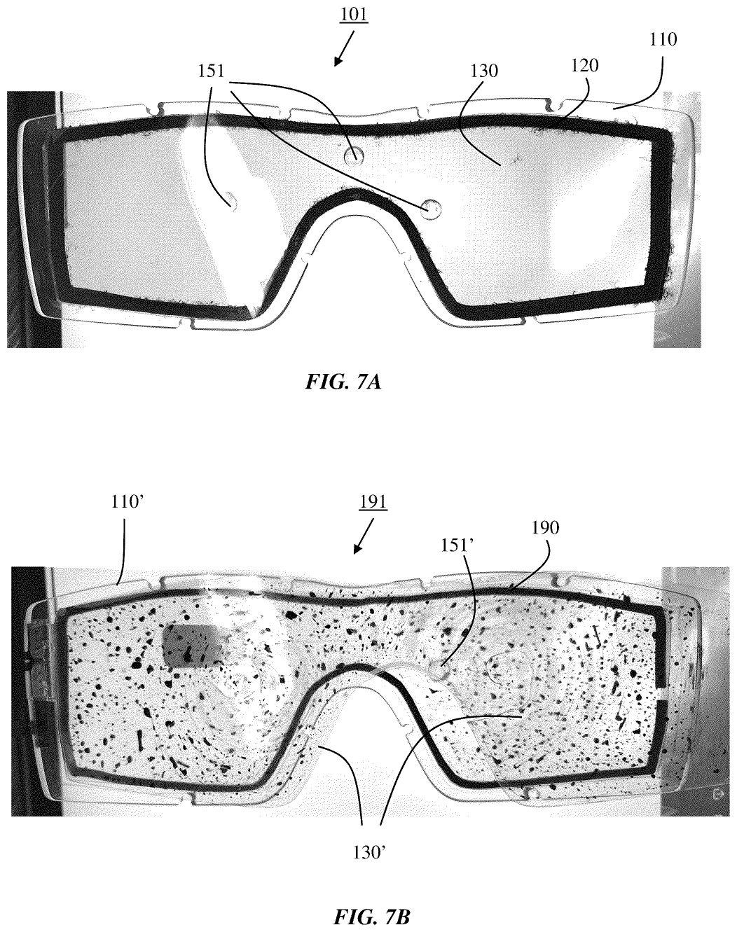

FIG. 7A is a photograph of an example lens structure after ballistics testing. Lens structure 101 includes a light transmissive front lens 110 spaced away from rear optical element 130 by a pressure-relieving attachment structure 120, as discussed elsewhere herein. In the present example, rear optical element 130 is an electronically active lens that includes a liquid crystal material. The pressure-relieving attachment structure 120 in this case is a HAL fastener. The front lens 110 is impact resistant. One can also see three impact dents 151 in the front lens. However, the lens structure stays intact and remains functional.

FIG. 7B is photograph a comparative lens structure 191 (not according to the present disclosure) after ballistic testing. Comparative lens structure 191 is like lens structure 101 except that a seal 190 was used that does not have substantial pressure-relieving properties. As a result of ballistic impact 151′ on the front lens 110′, the rear optical element 130′ became detached from the front lens (one can see that the rear optical element is no longer properly aligned with the front lens) and a seal between two substrates of the electronically active lens was compromised resulting in spatter of the liquid crystal material and a non-functional electronically active lens.

Optical Systems with Pressure-Relieving Structures

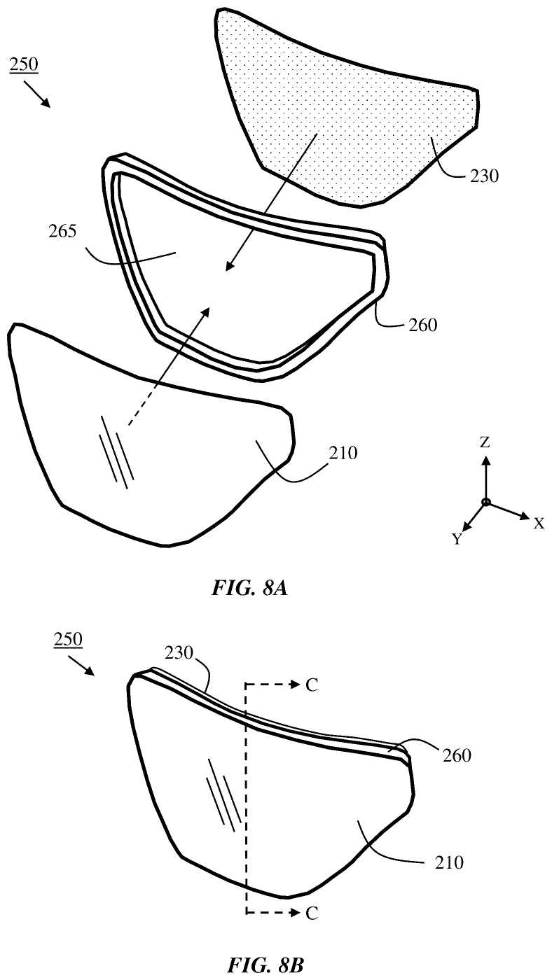

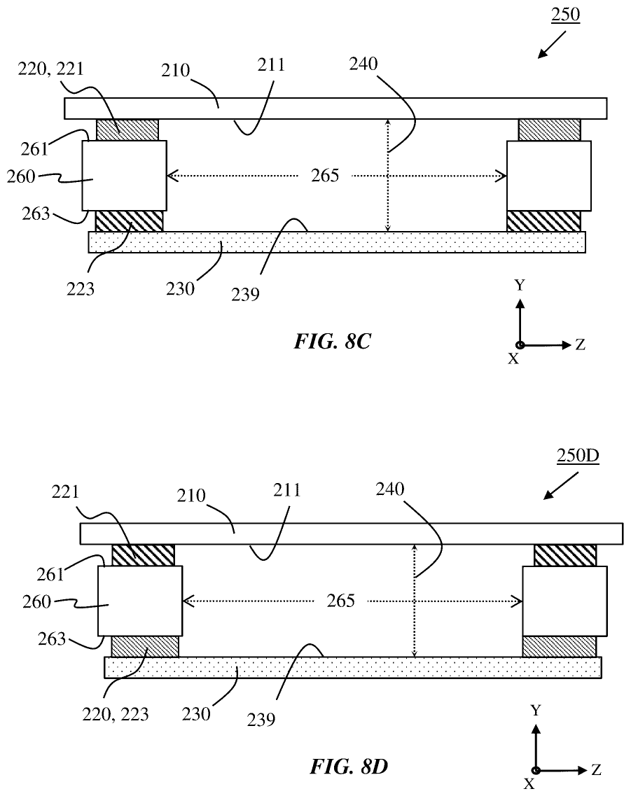

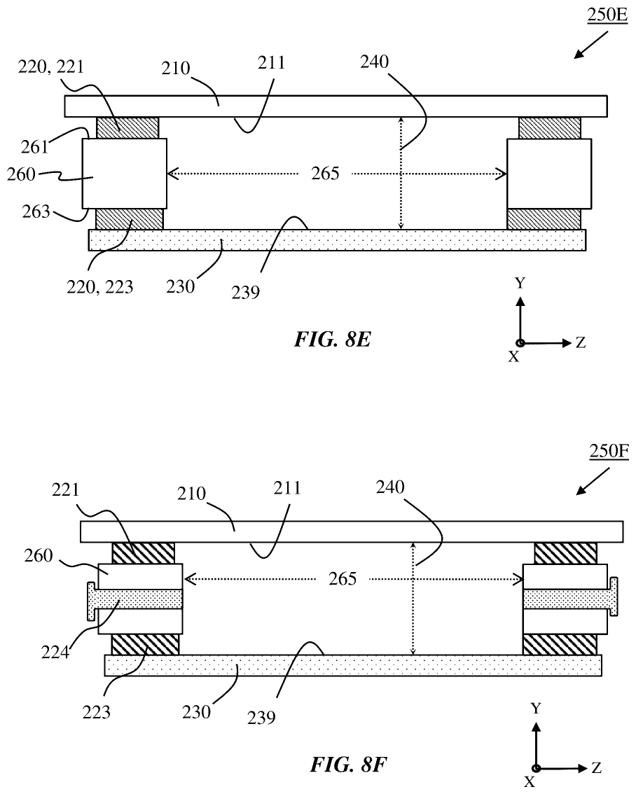

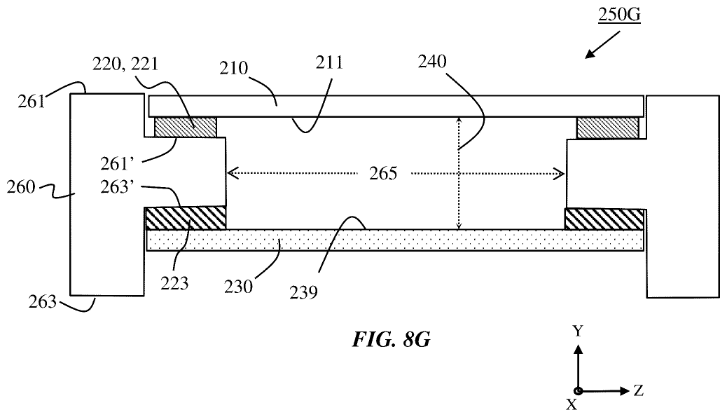

In some cases, rather than having the front lens directly attached to the rear optical element by a pressure-relieving attachment structure, these components may be attached to a frame and a pressure-relieving structure may be provided at the frame attachment or even in the frame itself. FIG. 8A is a perspective, expanded view of the components of a non-limiting example of an optical system 250. FIG. 8B is a perspective of the optical system and FIG. 8C is a cross-sectional view of the optical system along cutline C-C of FIG. 8B. Referring to all three figures, optical system 250 may include a frame 260 having a frame opening 265, a light transmissive front lens 210 extending across the frame opening 265 and attached to the frame 260 by a front attachment element 221. The front attachment element 221 may be provided around the periphery of the frame opening and interposed between the rear surface 211 of the front lens 210 and the frame 260, e.g., frame front surface 261. A rear optical element 230 extends across the frame opening 265 and is attached to the frame by a rear attachment element 223. Rear attachment element 223 may be provided around the periphery of the frame opening and interposed between the front surface 239 of the rear optical element 230 and the frame 260, e.g., frame rear surface 263. Front lens 210 and rear optical element 230 are separated by a gap 240. The properties of front lens 210, rear optical element 230, and gap 240 may be as described elsewhere with respect to front lens 110, rear optical element 130, and gap 140 of lens structure 101.

Optical system 250 includes at least one pressure-relieving structure that is responsive to changes in pressure within gap 240. In some examples, as shown in FIG. 8C, the pressure-relieving structure may be a pressure-relieving attachment structure 220 corresponding to front attachment element 221. A pressure-relieving attachment structure 220 may be as described elsewhere herein, e.g., with respect to any of pressure-relieving attachment structures 120, 320, 420, or 520. It is noted that, in examples using a HAL fastener, the front lens may be removable/replaceable. When an attachment element does not have substantial pressure-relieving properties, such as rear attachment element 223 shown in FIG. 8C, such attachment element may include an adhesive (e.g., a glue or epoxy), two-sided tape, a thermoplastic, or any other material or structure that can bond (optionally permanently) the rear optical element to the frame.

In some examples, the frame may be formed from a plastic, a polymer composite (e.g., polymer plus particles or fibers of a non-polymeric material such as glass, ceramic, metal, mineral, or the like), a metal, or a combination.

FIG. 8D is a cross-sectional view of another non-limiting example of an optical system 250D, where now the pressure-relieving attachment structure 220 is the rear attachment element 223 and the front attachment element 221 does not have substantial pressure-relieving properties. It is noted that, in examples using a HAL fastener as the pressure-relieving attachment structure 220, the rear optical element may be removable/replaceable.

FIG. 8E is a cross-sectional view of another non-limiting example of an optical system 250E where now both the front and rear attachment elements (221 and 223, respectively) include pressure-relieving attachment structures 220. It is noted that, in examples using a HAL fastener as the pressure-relieving attachment structure 220, the front lens and/or the rear optical element may be removable/replaceable.

FIG. 8F is a cross-sectional view of another non-limiting example of an optical system 250F where the pressure-relieving structure includes a pressure-relieving frame element 224. Pressure relieving frame element 224 is incorporated into frame 260 so that it is responsive to pressure changes within gap 240. For example, pressure-relieving element frame 224 may be a pressure-release valve.

Although FIGS. 8A-8F show the front lens and rear optical elements attached to front and back surfaces of the frame, respectively, one or both may instead be attached at a shelf structure within a frame recess. For example, FIG. 8G is a cross-sectional view of optical system 250G. In this case, the front lens 210 is attached to front shelf 261′ by front attachment element 221 (which in this figure is also a pressure-relieving attachment structure 220). The rear optical element 230 may be attached to rear shelf 263′ by rear attachment element 223. The recessed structure may in some cases help protect the lens, e.g., from impacts at the edge.

The advantages and benefits of a pressure-relieving structure to the optical system may be similar to those described with respect to the pressure-relieving attachment structure of the lens structure discussed elsewhere herein. Although electronically active lenses have been discussed primarily as a rear optical element, in some cases, the electronically active lens may be provided as the front lens, and the rear optical element may be another electronically active lens, a prescription lens, or any other optical element or lens discussed herein. Similarly, although prescription lenses have been discussed primarily as a rear optical element, in some cases, the prescription lens may be provided as the front lens, and the rear optical element may be an electronically active lens or any other optical element or lens discussed herein.

In some examples, the lens structure and/or optical system may be part of an eyewear system. In some cases, frame 260 may be incorporated into goggles, a visor, a helmet, or represent one portion of a frame for eyeglasses. Alternatively, the lens structure and/or optical system may be incorporated into windows, windshields, or sunroofs. In some cases, the lens structure and/or optical system may include extended reality technology or be incorporated into extended reality systems. For example, such extended reality (XR) technologies or systems, may include augmented reality (AR), virtual reality (VR), or mixed reality (MR), or some combination.

### Enumerated Aspects

Still further non-limiting examples of the present disclosure include the following enumerated aspects.

Enumerated aspect 1. A lens structure (101) including a light transmissive front lens (110) spaced away from a rear optical element (130) by a pressure-relieving attachment structure (120) provided around a viewing area perimeter of a front surface (139) of the rear optical element, wherein the pressure-relieving attachment structure further contacts a rear surface (111) of the front lens to form a gap (140) between the front lens and the rear optical element.

Enumerated aspect 2. The lens structure of enumerated aspect 1, wherein the pressure-relieving attachment structure includes a hook-and-loop fastener.

Enumerated aspect 3. The lens structure of enumerated aspect 1 or 2, wherein the pressure-relieving attachment structure includes a gas-venting structure.

Enumerated aspect 4. The lens structure according to any of the preceding enumerated aspects, wherein the pressure-relieving attachment structure includes a pressure-flexing structure.

Enumerated aspect 5. The lens structure according to any of the preceding enumerated aspects, wherein the gap is in a range of 0.5-5 mm.

Enumerated aspect 6. The lens structure according to any of the preceding enumerated aspects, wherein an area formed by the perimeter is in a range of 10-70 cm².

Enumerated aspect 7. The lens structure according to any of the preceding enumerated aspects, wherein the front lens is impact resistant.

Enumerated aspect 8. The lens structure according to any of the preceding enumerated aspects, wherein the rear optical element is flexible.

Enumerated aspect 9. The lens structure according to any of the preceding enumerated aspects, wherein the front lens or the rear optical element includes an electronically active lens.

Enumerated aspect 10. The lens structure of enumerated aspect 9, wherein the electronically active lens includes a liquid crystal device, an electrochromic optical device, a reversible metal electrodeposition device, a polarizer-based electro optical device, a graduated electrooptic device, or an electronic lensing device.

Enumerated aspect 11. The lens structure of enumerated aspect 10, wherein the liquid crystal device includes a guest-host mixture including a liquid crystal host and a dichroic or photodichroic dye guest.

Enumerated aspect 12. The lens structure according to any of the preceding enumerated aspects, wherein the front lens or the rear optical element includes a prescription lens.

Enumerated aspect 13. The lens structure according to any of the preceding enumerated aspects, the front lens or the rear optical element absorbs or reflects narrowband radiation.

Enumerated aspect 14. The lens structure according to any of the preceding enumerated aspects, wherein the front lens or the rear optical element includes a polarizer.

Enumerated aspect 15. The lens structure according to any of the preceding enumerated aspects, wherein the front surface of the rear optical element includes an antifog coating.

Enumerated aspect 16. The lens structure according to any of the preceding enumerated aspects, wherein the rear surface of the front lens includes an antifog coating.

Enumerated aspect 17. A lens structure including a light transmissive front lens (110) spaced away from a rear optical element (130) by a hook-and-loop (“HAL”) fastener (320) provided around a viewing area perimeter of a front surface (139) of the rear optical element, wherein the HAL fastener further contacts a rear surface (111) of the front lens to form a gap (140) between the front lens and the rear optical element.

Enumerated aspect 18. The lens structure of enumerated aspect 17, wherein the rear optical element includes a prescription lens or an electronically active lens.

Enumerated aspect 19. The lens structure of enumerated aspect 17 or 18, wherein the front lens is impact resistant.

Enumerated aspect 20. An eyewear system including at least one lens structure according to any of enumerated aspects 1-19, optionally wherein the eyewear system includes extended reality technology.

Enumerated aspect 21. The eyewear system of enumerated aspect 20, further including a frame to support the lens structure.

Enumerated aspect 22. The eyewear system of enumerated aspect 21, wherein the lens structure is attached to the frame by the front lens.

Enumerated aspect 23. The eyewear system of enumerated aspect 21 or 22, wherein the lens structure is attached to the frame by the rear optical element.

Enumerated aspect 24. The eyewear system according to any of enumerated aspects 20-23, wherein the front lens is impact resistant and the eyewear system meets the requirements of the MIL-PRF-31013 standard, the MIL-DTL-43511D standard, or the ANSI Z87.1 standard.

Enumerated aspect 25. An optical system (250) including: a frame (260) having a frame opening (265); a light transmissive front lens (210) extending across the frame opening and attached to the frame by a front attachment element (221); a rear optical element (230) extending across the frame opening and attached to the frame by a rear attachment element (223) and spaced away from the front lens by a gap (240); and a pressure-relieving structure (221) responsive to pressure changes within the gap, wherein the pressure-relieving structure includes: i) a pressure-relieving attachment structure corresponding to the front attachment element; ii) a pressure-relieving attachment structure corresponding to the rear attachment element; iii) a pressure-relieving frame element; or iv) any combination of (i)-(iii).

Enumerated aspect 26. The optical system of enumerated aspect 25, wherein at least one pressure-relieving attachment structure includes a hook-and-loop fastener.

Enumerated aspect 27. The optical system of enumerated aspect 25 or 26, wherein the pressure-relieving structure includes a gas-venting structure.

Enumerated aspect 28. The optical system according to any of enumerated aspects 25-27, wherein at least one pressure-relieving attachment includes a pressure-flexing structure.

Enumerated aspect 29. The optical system according to any of enumerated aspects 25-28, wherein the gap is in a range of 0.5-5 mm.

Enumerated aspect 30. The optical system according to any of enumerated aspects 25-29, wherein the frame is made from a plastic, a polymer composite, metal, or any combination thereof.

Enumerated aspect 31. The optical system according to any of enumerated aspects 25-30, wherein the front lens is attached to a front surface of the frame or to a front shelf within the frame.

Enumerated aspect 32. The optical system according to any of enumerated aspects 25-31, wherein the rear optical element is attached to a rear surface of the frame or to a rear shelf within the frame.

Enumerated aspect 33. The optical system according to any of enumerated aspects 25-32, wherein the front lens is impact resistant.

Enumerated aspect 34. The optical system according to any of enumerated aspects 25-33, wherein the rear optical element is flexible.

Enumerated aspect 35. The optical system according to any of enumerated aspects 25-34, wherein the front lens or the rear optical element includes an electronically active lens.

Enumerated aspect 36. The optical system of enumerated aspect 35, wherein the electronically active lens includes a liquid crystal device, an electrochromic optical device, a reversible metal electrodeposition device, a polarizer-based electro optical device, a graduated electrooptic device, or an electronic lensing device.

Enumerated aspect 37. The optical system of enumerated aspect 36, wherein the liquid crystal device includes a guest-host mixture including a liquid crystal host and a dichroic or photodichroic dye guest.

Enumerated aspect 38. The optical system according to any of enumerated aspects 25-37, wherein the front lens or the rear optical element includes a prescription lens.

Enumerated aspect 39. The optical system according to any of enumerated aspects 25-38, the front lens or rear optical element absorbs or reflects narrowband radiation.

Enumerated aspect 40. The optical system according to any of enumerated aspects 25-39, wherein the front lens or rear optical element includes a polarizer.

Enumerated aspect 41. The optical system according to any of enumerated aspects 25-40, wherein the front surface of the rear optical element includes an antifog coating.

Enumerated aspect 42. The optical system according to any of enumerated aspects 25-41, wherein the rear surface of the front lens includes an antifog coating.

Enumerated aspect 43. An optical system including: a frame having a frame opening; a light transmissive front lens extending across the frame opening and attached to the frame by a front attachment element; and a rear optical element extending across the frame opening and attached to the frame by a rear attachment element and spaced away from the front lens by a gap, wherein: i) the front attachment element includes a HAL fastener; ii) the rear attachment element includes a HAL fastener; or iii) both (i) and (ii).

Enumerated aspect 44. The optical system of enumerated aspect 43, wherein the rear optical element includes a prescription lens or an electronically active lens.

Enumerated aspect 45. The optical system of enumerated aspect 43 or 44, wherein the front lens is impact resistant.

Enumerated aspect 46. An eyewear system including the optical system according to any of enumerated aspects 25-45, optionally wherein the eyewear system includes extended reality technology.

Enumerated aspect 47. The eyewear system of enumerated aspect 46, wherein the front lens is impact resistant and the eyewear system meets the requirements of the MIL-PRF-31013 standard, the MIL-DTL-43511D standard, or the ANSI Z87.1 standard.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

In the preceding description, for the purposes of explanation, numerous details have been set forth in order to provide an understanding of various embodiments of the present technology. It will be apparent to one skilled in the art, however, that certain embodiments may be practiced without some of these details, or with additional details.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Additionally, details of any specific embodiment may not always be present in variations of that embodiment or may be added to other embodiments.

As used herein, a phrase that recites a range of values is inclusive of the end values, for example, “between X and Y,” “range of X to Y,” “from X to Y,” includes X and Y, or the phrase “up to Y” includes Y. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a method” includes a plurality of such methods and reference to “the layer” includes reference to one or more layers and equivalents thereof known to those skilled in the art, and so forth. The invention has now been described in detail for the purposes of clarity and understanding. However, it will be appreciated that certain changes and modifications may be practiced within the scope of the appended claims.

All publications, patents, and patent applications cited herein are hereby incorporated by reference in their entirety for all purposes. None is admitted to be prior art.

We claim:

1. A lens structure comprising a light transmissive front lens spaced away from a rear optical element by a pressure-relieving attachment structure provided around a viewing area perimeter of a front surface of the rear optical element, wherein the pressure-relieving attachment structure further contacts a rear surface of the front lens to form a gap between the front lens and the rear optical element.

2. The lens structure of claim 1, wherein the pressure-relieving attachment structure comprises a hook-and-loop fastener.

3. The lens structure of claim 1, wherein the pressure-relieving attachment structure comprises a gas-venting structure or a pressure-flexing structure.

4. The lens structure of claim 1, wherein the pressure-relieving attachment structure comprises a pressure-flexing structure.

5. The lens structure of claim 1, wherein the gap is in a range of 0.5-5 mm.

6. The lens structure of claim 1, wherein an area formed by the perimeter is in a range of 10-70 cm².

7. The lens structure of claim 1, wherein the front lens is impact resistant.

8. The lens structure of claim 1, wherein the rear optical element is flexible.

9. The lens structure of claim 1, wherein the rear optical element comprises an electronically active lens.

10. The lens structure of claim 9, wherein the electronically active lens comprises a liquid crystal device, an electrochromic optical device, a reversible metal electrodeposition device, a polarizer-based electro optical device, a graduated electrooptic device, or an electronic lensing device.

11. The lens structure of claim 1, wherein the rear optical element comprises a prescription lens.

12. The lens structure of claim 1, the front lens or the rear optical element absorbs or reflects narrowband radiation or wherein the front lens or the rear optical element comprises a polarizer.

13. The lens structure of claim 1, wherein i) the front surface of the rear optical element comprises an antifog coating, ii) the rear surface of the front lens comprises an antifog coating, or iii) both (i) and (ii).

14. An eyewear system comprising the lens structure of claim 1.

15. The eyewear system of claim 14, wherein the front lens is impact resistant and the eyewear system meets the requirements of the MIL-PRF-31013 standard, the MIL-DTL-43511D standard, or the ANSI Z87.1 standard.

16. An optical system comprising:
a frame having a frame opening;
a light transmissive front lens extending across the frame opening and attached to the frame by a front attachment element;
a rear optical element extending across the frame opening and attached to the frame by a rear attachment element and spaced away from the front lens by a gap; and
a pressure-relieving structure responsive to pressure changes within the gap, wherein the pressure-relieving structure includes:
i) a pressure-relieving attachment structure corresponding to the front attachment element;
ii) a pressure-relieving attachment structure corresponding to the rear attachment element;
iii) a pressure-relieving frame element; or
iv) any combination of (i)-(iii).

17. The optical system of claim 16, wherein at least one pressure-relieving attachment structure comprises a hook-and-loop fastener, a gas-venting structure, a pressure-flexing structure, or any combination thereof.

18. The optical system of claim 16, wherein the gap is in a range of 0.5-5 mm.

19. The optical system of claim 16, wherein the front lens is impact resistant.

20. The optical system of claim 16, wherein the rear optical element comprises a prescription lens or an electronically active lens, and wherein the electronically active lens comprises a liquid crystal device, an electrochromic optical device, a reversible metal electrodeposition device, a polarizer-based electro optical device, a graduated electrooptic device, or an electronic lensing device.
